(19) United States
(12) Patent Application Publication
(10) Pub. No.: US 2005/0227930 A1
(43) Pub. Date: Oct. 13, 2005

(54) Cholesterol lowering combination

(75) Inventor: Najla Guthrie, London (CA)

Correspondence Address: DAVIDSON, DAVIDSON & KAPPEL, LLC, 485 SEVENTH AVENUE, 14TH FLOOR, NEW YORK, NY, 10018, US

(73) Assignee: KGK Synergize Inc., London (CA)

(21) Appl. No.: 11/102,278

(22) Filed: Apr. 8, 2005

Related U.S. Application Data
(60) Provisional application No. 60/560,284, filed on Apr. 8, 2004.

(51) Int. Cl. A61K 31/7048; A61K 31/353; A61K 31/355; A61K 31/56

(52) U.S. Cl. 514/27; 514/171; 514/456; 514/458

(57) ABSTRACT

Compositions and methods for the treatment for the prevention and treatment of hypercholesterolemia and atherosclerosis are described. Individuals at a high risk of developing or having hypercholesterolemia and atherosclerosis may be treated with an effective dose of a composition comprising at least one citrus flavonoid, at least one tocotrienol, and at least one phytosterol or phytosterol ester.

### BACKGROUND OF THE INVENTION

1. Field of the Invention

The present invention relates to compositions and methods for the treatment and prevention of hypercholesterolemia and atherosclerosis.

2. Description of the Prior Art

In the United States, the complications of arteriosclerosis account for about one half of all deaths and for about one third of deaths in persons between 35 and 65 years of age. Atherosclerosis, or the development of atheromatous plaques in large and medium-sized arteries, is the most common form of arteriosclerosis. Many factors are associated with the acceleration of atherosclerosis, regardless of the underlying primary pathogenic change, for example, age, elevated plasma cholesterol level, high arterial blood pressure, cigarette smoking, reduced high-density lipoprotein (HDL) cholesterol level, or family history of premature coronary artery disease.

Elevated levels of blood cholesterol are known to be one of the major risk factors associated with coronary heart disease, the leading cause of death in North America. Dietary intervention has been proven to play an important role in prevention and treatment of hypercholesterolemia. Current dietary recommendations focus on reduced intake of saturated fat and cholesterol but numerous studies also demonstrated a role of other common macro- and micronutrients, such as carbohydrates, protein and vitamins, in modulation of cholesterolemic responses (Charleux, J. L. Nutr. Rev. 1996 54: S109-S114). However, during recent years, a growing interest has also been shown in investigating possible cardioprotective effects of plant-derived food products and their minor nutritive and non-nutritive constituents (Cook, N. C.; Samman, S. J. Nutr. Biochem. 1996, 7:66-76).

The risk of death from coronary artery disease has a continuous and graded relationship to total serum cholesterol levels greater than 180 mg/dl (Stamler, J. Et al., 1986, JAMA 2546:2823). Approximately one third of adults in the United States have levels that exceed 240 mg/dl and, therefore, have a risk of coronary artery disease that is twice that of people with cholesterol levels lower than 180 mg/dL.

The lipid transport system involves lipoproteins which transport cholesterol and triglycerides from sites of absorption and synthesis to sites of utilization. The lipoprotein surface coat contains the free cholesterol, phospholipids, and apolipoproteins, thus permitting these particles to be miscible in plasma as they transport their hydrophobic cargo. Apolipoprotein B (apo-B) is the principal protein of the cholesterol-carrying low density lipoproteins (LDL) and is the determinant for cellular recognition and uptake of LDL by the high affinity LDL receptor. Binding of apo-B to LDL receptors results in internalization and degradation of LDL, promoting the clearance of LDL from plasma and regulating intracellular cholesterol handling and biosynthesis.

Acceleration of atherosclerosis is principally correlated with elevation of LDL, or beta fraction, which is rich in cholesterol but poor in triglycerides. Elevation of HDL or alpha fraction, has a negative correlation with atherosclerosis (Castelli, W. P. et al., 1986, JAMA 256:2835). HDL exerts a protective effect and the ratio of total cholesterol to HDL cholesterol is a better predictor of coronary artery disease than the level of either alone. Total cholesterol levels are classified as being desirable (<200 mg/dl), borderline high (200-239 mg/dl), or high (>240 mg/dl) (Report of the National Education Program Expert Panel on Detection, Evaluation, and Treatment of High Blood Cholesterol in Adults, 1988, Arch. Intern. Med. 148:36).

Advances in the study of cholesterol metabolism and coronary disease have initiated an era of increased emphasis on preventive therapy. New guidelines for the detection and treatment of high blood cholesterol in adults recommend that patients with high cholesterol levels or with borderline-high levels and two or more additional risk factors should have a measurement of LDL. LDL cholesterol levels are then classified as borderline-high risk (130-159 mg/dl) or high risk (≧160 mg/dl). Dietary treatment is recommended for those patients with high-risk levels of LDL and for those with borderline-high risk levels who have two or more additional risk factors. Drug treatment is recommended for all patients with LDL levels greater than 189 mg/dl and for those patients with LDL cholesterol levels between 159 and 189 mg/dl who have two or more additional risk factors. Among the many drugs that have been used to reduce serum cholesterol levels are cholestyramine, colestipol, clofibrate, gemfibrozil and lovastatin.

Phytosterols and Phytosterol Esters

U.S. Pat. No. 5,952,393 teaches the use of compositions comprising phytosterols and policosanol as a means of lowering cholesterol. Phytosterols are compounds that are structurally similar in nature to cholesterol. As such, these compounds can compete with cholesterol for binding to receptor sites thus inhibiting the absorption of cholesterol by the body. While cholesterol is readily absorbed, phytosterols have either a low level of absorption or are unable to be absorbed by the body. Phytosterols also have been found to compete with cholesterol for interaction with the enzyme cholesterol esterase. Cholesterol esterase is an enzyme that catalyses the hydrolysis of cholesterol in order to generate cholesterol plus a fatty acid anion. Broken down cholesterol can than be absorbed by microvilli that line the walls of the small intestine. Through their interaction with cholesterol esterase, phytosterols interfere with the intestinal absorption of cholesterol. PCT Patent Application No. WO 01/32031 teaches a composition comprising a phytosterol or a phytosterol ester and a surfactant for reducing cholesterol. Phytosterol esters are phytosterols that have been modified to form a plant phytosterol ester derivative. Phytosterol esters are well known in the art as described in “Analysis of Sterols” by L. J. Goad and Akihisa which is herein incorporated by reference.

Citrus Flavonoids

Flavonoids are polyphenolic compounds that occur ubiquitously in plant foods especially in orange, grapefruit and tangerine. Epidemiological studies have shown that flavonoids present in the Mediterranean diet reduce the risk of death from coronary heart disease (Hertog, M. G. Et al., 1993, Lancet: 342, 1007-1011). Soybean isoflavones for example, genistein, which is a minor component of soy protein preparations may have cholesterol-lowering effects (Kurowska, E. M. et al., 1990, J. Nutr. 120:831-836). The flavonoids present in citrus juices such as orange and grapefruit include, but are not limited to, hesperetin and naringenin respectively. The flavonoids present in tangerine include, but are not limited to tangeretin or nobiletin. The aforementioned flavonoids are illustrated below:

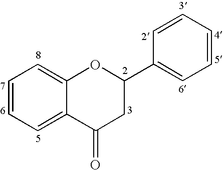

| | 5 | 7 | 3″ | 4″ |
|---|---|---|---|---|
| HESPERETIN | OH | OH | OH | OCH₃ |
| NARINGENIN | OH | OH | — | OH |

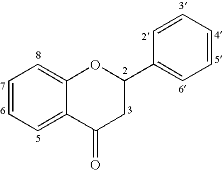

| | 5 | 6 | 7 | 8 | 4″ | 5″ |
|---|---|---|---|---|---|---|
| TANGERETIN | O CH₃ | O CH₃ | O CH₃ | O CH₃ | O CH₃ | — |
| NOBILETIN | O CH₃ | O CH₃ | O CH₃ | O CH₃ | O CH₃ | O CH₃ |

Tocotrienols

Tocotrienols are present in palm oil and are a form of vitamin E having an unsaturated side chain. They include, but are not limited to alpha-tocotrienol, gamma-tocotrienol or delta-tocotrienol as illustrated below. Tocotrienols have been found to inhibit LDL cholesterol synthesis, and the inhibit the synthesis of Apo B.

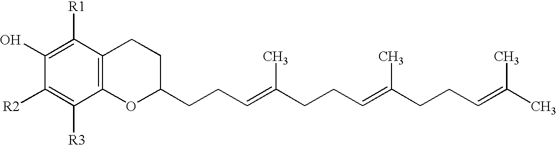

| | R1 | R2 | R3 |
|---|---|---|---|
| α-tocotricnol | CH₃ | CH₃ | CH₃ |
| γ-tocotricnol | H | CH₃ | CH₃ |
| δ-tocotricnol | H | H | CH₃ |

U.S. Pat. No. 6,251,400, and U.S. Patent Application No. 2001/0055627 teach cholesterol lowering compositions comprising tocotrienols and flavonoids.

The present invention seeks to provide improved compositions for treating or preventing increases in blood cholesterol levels.

All publications, patents and patent applications are herein incorporated by reference in their entirety to the same extent as if each individual publication, patent or patent application was specifically and individually indicated to be incorporated by reference in its entirety.

### SUMMARY OF THE INVENTION

In one embodiment the present invention provides a pharmaceutical composition for the prevention and treatment of hypercholesterolemia and atherosclerosis, the composition comprising a cholesterol lowering amount of:
at least one citrus flavonoid;
at least one phytosterol or phytosterol ester; and
at least one tocotrienol.

In one embodiment the present invention provides a method of treating hypercholesterolemia or atherosclerosis in a mammal, the method comprising administration of a pharmaceutical composition comprising a cholesterol lowering amount of:
at least one citrus flavonoid;
at least one phytosterol or phytosterol ester; and
at least one tocotrienol to a mammal.

### DESCRIPTION OF THE PREFERRED EMBODIMENTS

In one embodiment the present invention provides a pharmaceutical composition for the prevention and treatment of hypercholesterolemia and atherosclerosis, the composition comprising a cholesterol lowering effective amount of:
at least one citrus flavonoid;
at least one phytosterol or phytosterol ester; and
at least one tocotrienol.

It is believed that the ability of flavonoids, and/or tocotrienols to lower cholesterol, to inhibit liver cholesterol synthesis, and inhibit LDL cholesterol and apo-B synthesis, contributes to their effectiveness in the reduction of atherosclerosis and hypercholesterolemia and lowering the risk of cardiovascular disease. These possible mechanisms of action are in no way meant to limit the scope of the invention and are presented purely for illustrative purposes.

It is also believed that the ability of phytosterols or physterol esters to inhibit absorption of cholesterol, and to compete with cholesterol for the enzyme cholesterol esterase contributes to their effectiveness in the reduction of atherosclerosis and hypercholesterolemia and lowering the risk of cardiovascular disease. These possible mechanisms of action are in no way meant limit the scope of the invention and are presented purely for illustrative purpose.

The citrus flavonoids of the pharmaceutical preparation of the present invention include, but are not limited to, heptamethoxyflavone, sinensetin, 5-desmethylsinensetin, hesperidin, naringin, naringenin, hesperetin, nobiletin and tangeretin.

The tocotrienols of the pharmaceutical preparation of the present invention include, but are not limited to, alpha-tocotrienol, gamma-tocotrienol or delta-tocotrienol.

Although citrus flavonoids, tocotrienols, and phytosterols or phytosterol esters appear to lower cholesterol levels through different mechanisms, a combination of such compounds is believed to have a synergistic effect on lowering levels of cholesterol. Specifically, while phytosterols or phytosterol esters may lower cholesterol through direct competition for receptor binding sites and impeding cholesterol interaction with cholesterol esterase, and citrus flavonoids and tocotrienols may lower cholesterol by inhibiting liver cholesterol synthesis, LDL cholesterol, and apo-B synthesis, the combination of these three types of compounds may have a synergistic effect in lowering cholesterol beyond levels reached by use of one of these compounds individually. As such, a composition comprising a combination of these three compounds represents an improved treatment for hypercholesterolemia and atherosclerosis.

The combination of citrus flavonoids, tocotrienols, and phytosterols or phytosterol esters may be formulated, according to the present invention, into pharmaceutical preparations for administration to mammals for prevention and treatment of cardiovascular disease, hypercholesterolemia or atherosclerosis.

In a further embodiment, the composition of the present invention comprises at least one pharmaceutically acceptable adjuvant including but not limited to the group consisting of diluents, stabilizers, binders, buffers, lubricants, coating agents, preservatives, emulsifiers, and suspensing agents. An individual skilled in the art will recognize other acceptable adjuvants.

Many of the citrus flavonoids, tocotrienols, and phytosterol or phytosterol esters may be provided as compounds with pharmaceutically compatible counterions, a form in which they may be soluble.

In one embodiment, the present invention provides a method of treating hypercholesterolemia or atherosclerosis through the administration to an individual of a pharmaceutical composition as described above.

The therapeutic compounds or pharmaceutical compositions may be administered intravenously, intraperitoneally, subcutaneously, intramuscularly, intrathecally, orally, rectally, topically or in an aerosol form.

Formulations suitable for oral administration include liquid solutions of the active compound dissolved in diluents such as saline, water or PEG 400; capsules or tablets, each containing a predetermined amount of the active agent as solid, granules or gelatin; suspensions in a approximate medium; and emulsions.

Formulations suitable for parenteral administration include aqueous and non-aqueous isotonic sterile solutions, which contain buffers, antioxidants and preservatives. The formulations may be in unit dose or multi-dose sealed containers.

Patient dosages for oral administration of citrus flavonoids range from 1-5000 mg/day, commonly 1000-2000 mg/day, and typically from 500-1500 mg/day. Stated in terms of patient body weight, usual dosages range from 15-70 mg/kg/day, commonly from 15-30 mg/kg/day, typically from 7-21 mg/kg/day.

Patient dosages for oral administration of tocotrienols range from 1-1200 mg/day, commonly 1-100 mg/day, and typically from 1-60 mg/day. Stated in terms of patient body weight, usual dosages range from 0.01-20 mg/kg/day, commonly from 0.01-2.0 mg/kg/day, typically from 0.01 to 1/0 mg/kg/day.

Patient dosages for oral administration of phytosterol or phytosterol esters range from 1-3000 mg/day, commonly 1-2000 mg/day and typically from 1-1000 mg/day. Stated in terms of patient body weight, usual dosages range from 0.01-50 mg/kg/day, commonly from 0.01-35 mg/kg/day, typically from 0.01 to 20 mg/kg/day.

A variety of delivery systems for the pharmacological compounds may be employed, including, but not limited to, liposomes and emulsions. The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Although the invention has been described with reference to certain specific embodiments, various modifications thereof will be apparent to those skilled in the art without departing from the spirit and scope of the invention as outlined in the claims appended hereto.

1. A pharmaceutical composition for lowering cholesterol in mammals, the composition comprising a cholesterol lowering amount of:
at least one citrus flavonoid;
at least one phytosterol or phytosterol ester; and
at least one tocotrienol.

2. The pharmaceutical composition according to claim 1, wherein said at least one flavonoid is selected from one or more flavonoids of the group consisting of hesparidin, naringin, naringenin, hesperitin, nobiletin and tangeretin.

3. The pharmaceutical composition according to claim 1, wherein said at least one tocotrienol is selected from one or more tocotrienols of the group consisting of alpha-tocotrienol, gamma-tocotrienol and delta-tocotrienol.

4. The pharmaceutical composition according to claim 1 further comprising at least one pharmaceutically acceptable adjuvant.

5. The pharmaceutical composition according to claim 4 wherein said at least one pharmaceutically acceptable adjuvant is selected from the group consisting of diluents, stabilizers, binders, buffers, lubricants, coating agents, preservatives, emulsifiers, and suspension agents.

6-7. (canceled)

8. A method of treating hypercholesterolemia or atherosclerosis in a mammal, the method comprising administration of a pharmaceutical composition comprising a cholesterol lowering amount of:
at least one citrus flavonoid;
at least one phytosterol or phytosterol ester; and
at least one tocotrienol.

9. The method according to claim 8, wherein said at least one flavonoid is selected from one or more flavonoids of the group consisting of hesparidin, naringin, naringenin, hesperitin, nobiletin and tangeretin.

10. The method according to claim 8, wherein said at least one tocotrienol is selected from one or more tocotrienols of the group consisting of alpha-tocotrienol, gamma-tocotrienol and delta-tocotrienol.

11. The method according to claim 8 further comprising at least one pharmaceutically acceptable adjuvant.

12. The method according to claim 11 wherein said at least one pharmaceutically acceptable adjuvant is selected from the group consisting of diluents, stabilizers, binders, buffers, lubricants, coating agents, preservatives, emulsifiers, and suspension agents.

13. The method according to claim 12 wherein said administration is intravenous, intraperitoneally, subcutaneous, intramuscular, oral, rectal, topical or aerosol.
